SEQUENCE LISTING (2 sequences)

SEQ ID NO: 1 - protein, 140 aa, Homo sapiens
MRSPRMRVCAKSVLLSHWLFLAYVLMVCCKLMSASSQHLRGHAGHHQIKQGTCEVVAVHRCCNKNRIEERSQTVKCSCFPGQVAGTTRAQPSCVEASIVIQKWWCHMNPCLEGEDCKVLPDYSGWSCSSGNKVKTTKVTR

SEQ ID NO: 2 - DNA, 2294 bp, Homo sapiens
aatcagcgcgttccgcggcgggcccggcccctcctggtcagcgcgctagctgggctcggctccgcactgctagctgcgcgccgccctggacggggcgcaccgactgcgcgcgcggctgcgggcaaacatcgggagtcctgcctcagctgccgcttctccagcagcagcttcaggcttctcccgcaggagcttcgggcttctcctggtagagacgtgggaacttttcttctcctggcgaggctgcagaggtgatgggccgctcccggggctcccgcggggaggcggcacggtgagcgtcctcgggctccggtgcggcgatcagtacctagttccggacgcgccggtccgacttggatgccggctctagtcgagtcttctgagctacgataattttttggaacggcagaaatgattggttctagcaacagatgggaatttggagtcactctgaaatatatcctggaataagtgtgtttgactagaaccacatcttatgaggtccccaaggatgagagtctgtgctaagtcagtgttgctgtcgcactggctctttctagcctacgtgttaatggtgtgctgtaagctgatgtccgcctcaagccagcacctccggggacatgcaggtcaccaccaaatcaagcaagggacctgtgaggtggtcgccgtgcacaggtgctgcaataagaaccgcatagaagagcggtcacaaacggtcaagtgctcttgcttcccgggacaggtggcgggcacaactcgggctcaaccttcttgtgttgaagcttccattgtgattcagaaatggtggtgtcacatgaatccgtgtttggaaggagaggattgtaaagtgctgccagattactcaggttggtcctgtagcagtggcaataaagtcaaaactacgaaggtaacgcggtagcgaagagagaggtgtgcttcaatcctggaggggcagcaggaggcggagctcttttgcttggattcccatcatggcccctttgcagaaaattgtctaggatttcagcaacttcatatttgtatatgtgagctgtgagaggtggcattcacttaactggcccagccctctctgcttcgtgattttatttcattgaattataaccacaagccaccacccatttgacatcctctctggattcccaaggagcatacctccaaaatccgagaagagcaaatcagagtcttcaaaatggatcaccactaagggcatgttcattcttcactttctttctgcttttacaaaagaacttggatgtatgttccaaagggtcctcattctgttcctcttttgaacttttccttttgtccttgtattaaagtggttttaaaggggtctaaaaagattttggcaaaacatatttgcagatgtagattagctggtgaagaaaattactgctagagatcaactgattaactggtaaagaacgtttattttataacccttgaagaatagaaggacatagttggattattgtgtgtgcattgtatttttacttctattttttttttgctttccattttccagttagcagagataaaatgagagcgttttaacttcaatgtaccattttactgagtgctaaggaagcatatcaattccaatattttataaccaaagctctatcagaacatatttataaaacttgttggaatttttacggcttttgtgtagtcatgtaggtaaatcatttaaaatataaaacaatctcaatttagatcaagggttatttcttagatcaaatttatgccaattatatgaaaagattttaactccgagacaggagtctttcagtgctgaatttttagactgtaaatgagttcttcttaacttagctgtttccctacttctgtgacttctgtgttagccatcttatttctttaaaatctgagtcctgattggcttaatgattttgcagcagacatgtctccacatattctcaaatgctgtcatgcggaaacgtatgaaacagatgaagaatgactgacccagattttagatgtataatgttgttaaagtacatactactgtaaaaatatgggatgaattttatatattaagaaatgccaaaaacatagtttctgcaccaagttaattatccctgtcctttcacatttatagggggaaaataaatactttaatgttgtttatagcctaacagttatttgattttattcttgcagagggaatggaaaggaatggaaagatttgttggcgtaatttttgaatatttgttatgatcatatgaataagtaaaaaaattcatcctgctgatggcata

(19) United States
(12) Patent Application Publication
(10) Pub. No.: US 2023/0068400 A1
(43) Pub. Date: Mar. 2, 2023

(54) TAFA4 POLYPEPTIDE OR POLYNUCLEOTIDE FOR TREATING INFLAMMATORY DISEASE

(71) Applicants: INSERM (INSTITUT NATIONAL DE LA SANTÈ ET DE LA RECHERCHE MÉDICALE), Paris (FR); CENTRE NATIONAL DE LA RECHERCHE SCIENTIFIQUE (CNRS), Paris (FR); UNIVERSITÉ D'AIX MARSEILLE, Marseille Cedex 07 (FR)

(72) Inventors: Sophie UGOLINI, Marseille (FR); Jordi GOUILLY, Marseille (FR); Guillaume DEBROAS, Marseille (FR)

(21) Appl. No.: 17/760,007

(22) PCT Filed: Feb. 3, 2021
(86) PCT No.: PCT/EP2021/052548

§ 371 Date: Aug. 3, 2022

(30) Foreign Application Priority Data
Feb. 4, 2020 (EP) 20305098.4
Apr. 14, 2020 (EP) 20306202.1
Oct. 14, 2020 (EP) 20315160.0

(51) Int. Cl. A61K 38/19; A61P 31/14; C07K 14/52; C07K 14/705

(52) U.S. Cl. CPC A61K 38/195; A61P 31/14; C07K 14/521; C07K 14/70503; C07K 2319/30

(57) ABSTRACT

The present invention relates to methods and pharmaceutical composition for treating inflammatory diseases. Inflammation is a defence response to tissue damage or infections that requires tight regulation to prevent impaired healing and to avoid excessive damage and/or autoimmunity. Myeloid cells, including macrophages play a key role in tissue repair and undergo major functional changes during the healing processes, switching from an inflammatory state to a pro-repair phenotype. The inventors have found that TAFA4, a chemokine-like protein, has anti-inflammatory and pro-repair properties. This molecule regulates the phenotype of man monocytes and macrophages by promoting their anti-inflammatory and pro-repair functions. TAFA4 increases macrophage their phagocytic capacities and their production of the anti-inflammatory cytokine IL-10. By contrast, TAFA4 down-regulates the production of the pro-inflammatory cytokines IL-6, IL-113, and TNF-α by human macrophages. Importantly, the inventors have also found that a treatment with TAFA4 has anti-inflammatory effects in vivo and protects mice from LPS-induced endotoxic shock. This protective effect is associated with a reduction in inflammatory cytokine levels and an increase in EL-10 production. Finally, they found that TAFA4 can also exert its anti-inflammatory properties on peripheral blood mononuclear cells from COVID-19 patients, independently of the disease severity. Thus, the present invention relates to a TAFA4 polypeptide or a nucleic acid molecule encoding thereof for use in the treatment of inflammatory diseases.

### FIELD OF THE INVENTION

The present invention relates to methods and pharmaceutical composition for treating inflammatory disease.

### BACKGROUND OF THE INVENTION

Inflammation is a coordinated process designed by evolution to eliminate pathogens and enable healing. However, inflammatory processes are carefully regulated in the sense that when it is no longer necessary, it must be actively terminated to avoid tissue damage and/or autoimmunity. Macrophages play central roles in the progression of inflammatory diseases. They respond to environmental cues rapidly and they can shift their phenotype within a wide ranges in vivo [1]. Macrophage activation is not only an essential component of host defenses against microbial infection but is also closely associated with regulating tissue homeostasis in inflammatory diseases such as sepsis, rheumatoid arthritis and atherosclerosis. It is well-known that inflammatory macrophages induced by immune stimuli or specific microenvironment conditions have increased glycolytic metabolism and reduced mitochondrial respiration. Recent studies suggest that rewiring the metabolic pathway of activated macrophages can serve as therapeutic approaches to treat inflammatory diseases such as sepsis or lung inflammation caused by viruses [2;8-10]. Sepsis is a life-threatening condition that arises when the body's response to infection causes injury to its own tissues and organs. Despite advances in clinical therapy, severe sepsis and septic shock are still the leading cause of death in intensive care units. Among the most common bacterial causes of sepsis are Gram-negative bacilli. A major component of Gram-negative bacteria, LPS, induces the secretion and release of multiple proinflammatory mediators such as TNF-α, IL-lb, IL-6 mainly by macrophages. Rheumatoid arthritis (RA) is a well-known chronic inflammatory disease characterized by extensive synovitis [3]. Systemic inflammation is mediated by pro-inflammatory cytokines, which are mainly produced by macrophages and lymphocytes in synovial tissue. Atherosclerosis is a chronic inflammatory disease triggered by lipid accumulation in the arterial wall. Macrophages are fundamental contributors to the progression of atherosclerosis. In hypercholesterolemia, macrophages infiltrate the arterial intima and clear accumulated modified low-density lipoprotein (LDL), which transforms them into lipid-laden macrophages. These macrophages trigger the formation of plaques and serve as inflammatory mediators to promote plaque progression [4].

For instance, a wide range of viruses inducing upper and lower respiratory tract infections have been identified as causes of significant morbidity and mortality among infants and adults. These respiratory viruses include members of the Pneumoviridae family, including human respiratory syncytial virus (hRSV) type A and B, and human metapneumovirus (h1VIPV) type A and B; members of the Paramyxoviridae family, including parainfluenza virus type 3 (PIV-3) and measles virus; and members of the Coronaviridae family, including endemic human coronaviruses (HCoV-229E, HCoV-NL63, HCoV-0C43, and HCoV-HKU1); severe acute respiratory syndrome coronavirus (SARS-CoV) and Middle-East respiratory syndrome coronavirus (MERS-CoV). In particular, in late December 2019, a new betacoronavirus SARS-CoV-2 has emerged in Wuhan China [8-10]. The World Health Organization has named the severe pneumonia caused by this new coronavirus COVID-19 (for Corona Virus Disease 2019, WHO, 2020). Since its emergence, the SARS-CoV-2 has spread to 2132 countries across the five continents causing, at the time of the writing, about 1,436,198 human infections and 85,522 deaths (WHO, Apr. 9 2020). Most patients with COVID-19 exhibit mild to moderate symptoms, but approximately 15% progress to severe pneumonia and about 5% eventually develop acute respiratory distress syndrome (ARDS), septic shock and/or multiple organ failure

. Most patients with severe COVID-19 exhibit substantially elevated serum levels of pro-inflammatory cytokines including IL-6 and IL-1(3, as well as IL-2, IL-8, IL-17, G-CSF, GM-CSF, IP10, MCP1, MIP 1 a (also known as CCL3) and TNF, characterized as cytokine storm

.

TAFA4 is a member of the TAFA family which is composed of five highly homologous genes that encode small secreted proteins. These proteins contain conserved cysteine residues at fixed positions, and are distantly related to MIP- 1 alpha, a member of the CC-chemokine family. TAFA4 molecule is thus a chemokine-like protein [5] known to modulate injury-induced mechanical and chemical pain hypersensitivity in mice [6]. Recombinant TAFA4 was shown to be involved in human macrophage chemotaxis in vitro [7]. However, its potential role on human macrophages phenotype and functions in inflammatory disease has not yet been investigated.

### SUMMARY OF THE INVENTION

The present invention relates to a method for treating inflammatory disease in a subject in need thereof comprising administering to the subject a therapeutically effective amount of a TAFA4 polypeptide or a nucleic acid molecule encoding thereof. In particular, the present invention is defined by the claims.

### DETAILED DESCRIPTION OF THE INVENTION

In order to assess the role of TAFA4 in human macrophages phenotype and functions, the inventors have found that TAFA4 skews the phenotype and the effector functions of human macrophages towards an anti-inflammatory and pro-repair profile, characterized by an increased phagocytosis associated with IL-10 production. Importantly, they have also found that TAFA4 exert anti-inflammatory effects in vivo and protects mice from LPS-induced endotoxic shock. Thus, TAFA4 would be suitable for the treatment of inflammatory diseases.

Accordingly, the first object of the present invention relates to a method of treating an inflammatory disease in a subject in need thereof comprising administering to the subject a therapeutically effective amount of a TAFA4 polypeptide or a nucleic acid molecule encoding thereof.

In other word, the present invention relates to a TAFA4 polypeptide or a nucleic acid molecule encoding thereof for use in the treatment of inflammatory disease.

As used herein, the term “subject” denotes a mammal, such as a rodent, a feline, a canine, and a primate. Preferably, a subject according to the invention is a human.

As used herein, the expression “inflammatory disease′ is used herein in the broadest sense and includes all diseases and pathological conditions having etiologies associated with a systemic or local abnormal and/or uncontrolled inflammatory response. For instance, over-expression of proinflammatory cytokines without proper controls leads to a variety of inflammatory diseases and disorders. This term includes both acute inflammatory diseases and chronic inflammatory diseases.

In particular, the above-mentioned inflammatory diseases may be one or more selected from the group consisting of asthma, preperfusion injury, transplant rejection, sepsis, septic shock, arthritis, rheumatoid arthritis, acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), x-linked hyper IgM syndrome, sclerosis, systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis, inflammatory bowel disease (IBD), Crohn's disease, colitis, ulcerative colitis, colitis ulcerosa, microscopic colitis, collagenous colitis, colitis polyposa, necrotizing enterocolitis, transmural colitis, autoimmune inflammatory bowel disease, pyoderma gangrenosum, erythema nodosum, primary sclerosing cholangitis, episcleritis, respiratory distress syndrome, adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis, Rasmussen's encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN), idiopathic membranous GN or idiopathic membranous nephropathy, membrano- or membranous proliferative GN (A/IPGN), rapidly progressive GN, allergic conditions, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis, lymphomatoid granulomatosis, Wegener's granulomatosis, agranulocytosis, vasculitides, including vasculitis, large vessel vasculitis, polymyalgia rheumatica, giant cell (Takayasu's) arteritis, medium vessel vasculitis, Kawasaki's disease, polyarteritis nodosa, microscopic polyarteritis, CNS vasculitis, necrotizing, cutaneous, hypersensitivity vasculitis, systemic necrotizing vasculitis, and ANCA-associated vasculitis, such as Churg-Strauss vasculitis or syndrome (CSS), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigus, optionally pemphigus vulgaris, pemphigus foliaceus, pemphigus mucus-membrane pemphigoid, pemphigus erythematosus, autoimmune polyendocrinopathies, Reiter's disease or syndrome, immune complex nephritis, antibody-mediated nephritis, neuromyelitis optica, polyneuropathies, chronic neuropathy, IgM polyneuropathies, IgM-mediated neuropathy, thrombocytopenia, thrombotic thrombocytopenic purpura (TTP), idiopathic thrombocytopenic purpura (ITP), autoimmune orchitis and oophoritis, primary hypothyroidism, hypoparathyroidism, autoimmune thyroiditis, Hashimoto's disease, chronic thyroiditis (Hashimoto's thyroiditis); subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis, allergic encephalomyelitis, experimental allergic encephalomyelitis (EAE), myasthenia gravis, thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AGED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis, optionally benign monoclonal gammopathy or monoclonal garnmopathy of undetermined significance, MGUS, peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia greata, CREST syndrome (calcinosis, Raynaud's phenomenon, esophageal dysmotility, sclerodactyl), and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillo sis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis (e.g. chronic pancreatitis), polyradiculitis acuta, pyoderma gangrenosum, Quervain's thyreoiditis, acquired splenic atrophy, infertility due to antispermatozoan antobodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, nonalcoholic fatty liver disease and endometriosis.

In particular, the above-mentioned inflammatory diseases is a viral infection.

In particular, the above-mentioned inflammatory diseases is a viral infection which caused lung inflammation, such viral pneumonia.

In particular, the viral infection is caused by a virus selected from the group consisting of influenza virus (e.g., Influenza virus A, Influenza virus B), respiratory syncytial virus, adenovirus, metapneumovirus, cytomegalovirus, parainfluenza virus (e.g., hPIV-1, hPIV-2, hPIV-3, hPIV-4), rhinovirus, coxsackie virus, echo virus, herpes simplex virus, coronavirus (SARS-coronavirus such as SARS-Cov 1 or SARS-Cov 2), and smallpox.

In some embodiments, the viral infection may be due to a member of the Pneumoviridae, Paramyxoviridae and/or Coronaviridae families are in particular selected from the group consisting of upper and lower respiratory tract infections due to: human respiratory syncytial virus (hRSV), type A and type B, human metapneumovirus (hMPV) type A and type B; parainfluenza virus type 3 (PIV-3), measles virus, endemic human coronaviruses (HCoV-229E, -NL63, -0C43, and -HKU1), severe acute respiratory syndrome (SARS) and Middle-East respiratory syndrome (MERS) coronaviruses.

In particular, the above-mentioned inflammatory disease is Severe Acute Respiratory Syndrome (SARS).

In more particular, the above-mentioned inflammatory disease is COVID-19.

In some embodiment, the inflammatory disease is sepsis.

In some embodiment, the inflammatory disease is not an inflammatory skin disease. In some embodiment, the inflammatory disease is not acne, rosacea, folliculitis, perioral dermatitis, photodamage, photodermatitis, skin aging, psoriasis, ichtiosis, chronic wounds such as venous stasis ulcers or diabetic foot ulcers, bed sores, keratosis piralis, scars, including surgical and acne scars, sebaceous cysts, inflammatory dermatoses, post inflammatory hyperpigmentation, xerosis, pruritis, lichen planus, nodular prurigo, eczema, miliaria, scleroderma, atopic dermatitis, nephrogenic fibrosing dermopathy, mixed connective tissue disease, scleromyxedema, scleredema, keloid, sclerodactyly, or eosinophilic fasciitis.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “TAFA4 polypeptide” designates a polypeptide belonging to the family of TAFA chemokine-like proteins, preferably comprising the amino acid sequence of SEQ ID NO: 1 (which corresponds to the human TAFA4 amino acid sequence) and any natural variant thereof (e.g., variants present in other animal species, or variants as a result of polymorphism or splicing). Within the context of the present invention, the term “TAFA4 polypeptide” also includes any polypeptide comprising a sequence having at least 90% sequence identity to the sequence shown in SEQ ID NO: 1.

SEQ ID NO:1 >sp|Q96LR4|F19A4_HUMAN Protein
FAM19A4 OS=Homo sapiens OX=9606 GN=FAM19A4
PE=1 SV=1
MRSPRMRVCAKSVLLSHWLFLAYVLMVCCKLMSASSQHLRGHAGHHQIK
QGTCEWAVHRCCNKNRIEERSQTVKCSCFPGQVAGTTRAQPSCVEASIV
IQKWWCHMNPCLEGEDCKVLPDYSGWSCSSGNKVKTTKVTR

According to the invention a first amino acid sequence having at least 90% of identity with a second amino acid sequence means that the first sequence has 90; 91; 92; 93; 94; 95; 96; 97; 98; 99 or 100% of identity with the second amino acid sequence. Sequence identity is frequently measured in terms of percentage identity (or similarity or homology); the higher the percentage, the more similar are the two sequences. Methods of alignment of sequences for comparison are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. Appls Biosci., 8:155-165, 1992; and Pearson et al., Meth. Mol. Biol., 24:307-31, 1994). Altschul et al., Nat. Genet., 6:119-129, 1994, presents a detailed consideration of sequence alignment methods and homology calculations. By way of example, the alignment tools ALIGN (Myers and Miller, CABIOS 4:11-17, 1989) or LFASTA (Pearson and Lipman, 1988) may be used to perform sequence comparisons (Internet Program® 1996, W. R. Pearson and the University of Virginia, fasta20u63 version 2.0u63, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

In some embodiment, the TAFA4 polypeptide of the invention comprises a sequence as set forth in SEQ ID NO: 1.

The polypeptides of the invention may be produced by any suitable means, as will be apparent to those of skill in the art. In order to produce sufficient amounts of polypeptides or functional equivalents thereof for use in accordance with the present invention, expression may conveniently be achieved by culturing under appropriate conditions recombinant host cells containing the polypeptide of the invention. In particular, the polypeptide is produced by recombinant means, by expression from an encoding nucleic acid molecule. Systems for cloning and expression of a polypeptide in a variety of different host cells are well known. When expressed in recombinant form, the polypeptide is in particular generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli.

In some embodiments, the TAFA4 polypeptide is fused to an immunoglobulin constant domain to constitute an immunoadhesin.

As used herein, the term “immunoadhesin” designates antibody-like molecules which combine the binding specificity of a heterologous protein (an “adhesin”) with the effector functions of immunoglobulin constant domains. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

The immunoglobulin sequence typically, but not necessarily, is an immunoglobulin constant domain (Fc region). Immunoadhesins can possess many of the valuable chemical and biological properties of human antibodies. Since immunoadhesins can be constructed from a human protein sequence with a desired specificity linked to an appropriate human immunoglobulin hinge and constant domain (Fc) sequence, the binding specificity of interest can be achieved using entirely human components. Such immunoadhesins are minimally immunogenic to the patient, and are safe for chronic or repeated use.

The polypeptides of the invention can exhibit post-translational modifications, including, but not limited to glycosylations, (e.g., N-linked or O-linked glycosylations), myristylations, palmitylations, acetylations and phosphorylations (e.g., serine/threonine or tyrosine).

In specific embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the polypeptide or fragment of the polypeptide described herein for therapeutic delivery.

As used herein, the term “nucleic acid molecule” has its general meaning in the art and refers to a DNA or RNA molecule. However, the term captures sequences that include any of the known base analogues of DNA and RNA such as, but not limited to 4-acetylcytosine, 8-hydroxy-N6-methyl adenosine, aziridinyl cyto sine, pseudoi socytosine, 5-(carboxyhydroxylm ethyl) uracil, 5-fiuorouracil, 5-b ro mouracil, 5-carboxymethylaminomethyl-2-thiouracil, 5-carboxymethyl- aminomethyluracil, dihydrouracil, inosine, N6-isopentenyladenine, 1-methyladenine, 1-methylpseudouracil, 1-methylguanine, 1- methylinosine, 2,2-dimethylguanine, 2-methyladenine, 2-methylguanine, 3-methylcytosine, 5- methylcytosine, N6-methyladenine, 7-methylguanine, 5-methylaminomethyluracil, 5-methoxyamino-methyl-2-thiouracil, beta-D-mannosylqueosine, 5′-methoxycarbonylmethyluracil, 5-methoxyuracil, 2-methylthio-N6-i sop entenyl adenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

In some embodiments, the nucleic acid molecule of the present invention comprises a nucleic acid sequence having has at least 70% identity with the nucleic acid sequence as set forth in SEQ ID NO:2. According to the invention a first nucleic acid sequence having at least 70% identity with a second nucleic acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; 99 or 100% identity with the second nucleic acid sequence.

```
SEQ ID NO:2
      1 aatcagcgcg ttccgcggcg ggcccggccc ctcctggtca gcgcgctagc tgggctcggc
    61 tccgcactgc tagctgcgcg ccgccctgga cggggcgcac cgactgcgcg cgcggctgcg
  121 ggcaaacatc gggagtcctg cctcagctgc cgcttctcca gcagcagctt caggcttctc
  181 ccgcaggagc ttcgggcttc tcctggtaga gacgtgggaa cttttcttct cctggcgagg
  241 ctgcagaggt gatgggccgc tcccggggct cccgcgggga ggcggcacgg tgagcgtcct
  301 cgggctccgg tgcggcgatc agtacctagt tccggacgcg ccggtccgac ttggatgccg
  361 gctctagtcg agtcttctga gctacgataa ttttttggaa cggcagaaat gattggttct
  421 agcaacagat gggaatttgg agtcactctg aaatatatcc tggaataagt gtgtttgact
  481 agaaccacat cttatgaggt ccccaaggat gagagtctgt gctaagtcag tgttgctgtc
  541 gcactggctc tttctagcct acgtgttaat ggtgtgctgt aagctgatgt ccgcctcaag
  601 ccagcacctc cggggacatg caggtcacca ccaaatcaag caagggacct gtgaggtggt
  661 cgccgtgcac aggtgctgca ataagaaccg catagaagag cggtcacaaa cggtcaagtg
  721 ctcttgcttc ccgggacagg tggcgggcac aactcgggct caaccttctt gtgttgaagc
  781 ttccattgtg attcagaaat ggtggtgtca catgaatccg tgtttggaag gagaggattg
  841 taaagtgctg ccagattact caggttggtc ctgtagcagt ggcaataaag tcaaaactac
  901 gaaggtaacg cggtagcgaa gagagaggtg tgcttcaatc ctggaggggc agcaggaggc
  961 ggagctcttt tgcttggatt cccatcatgg cccctttgca gaaaattgtc taggatttca
1021 gcaacttcat atttgtatat gtgagctgtg agaggtggca ttcacttaac tggcccagcc
1081 ctctctgctt cgtgatttta tttcattgaa ttataaccac aagccaccac ccatttgaca
1141 tcctctctgg attcccaagg agcatacctc caaaatccga gaagagcaaa tcagagtctt
1201 caaaatggat caccactaag ggcatgttca ttcttcactt tctttctgct tttacaaaag
1261 aacttggatg tatgttccaa agggtcctca ttctgttcct cttttgaact tttccttttg
1321 tccttgtatt aaagtggttt taaaggggtc taaaaagatt ttggcaaaac atatttgcag
1381 atgtagatta gctggtgaag aaaattactg ctagagatca actgattaac tggtaaagaa
1441 cgtttatttt ataacccttg aagaatagaa ggacatagtt ggattattgt gtgtgcattg
1501 tatttttact tctatttttt ttttgctttc cattttccag ttagcagaga taaaatgaga
1561 gcgttttaac ttcaatgtac cattttactg agtgctaagg aagcatatca attccaatat
1621 tttataacca aagctctatc agaacatatt tataaaactt gttggaattt ttacggcttt
1681 tgtgtagtca tgtaggtaaa tcatttaaaa tataaaacaa tctcaattta gatcaagggt
1741 tatttcttag atcaaattta tgccaattat atgaaaagat tttaactccg agacaggagt
1801 ctttcagtgc tgaattttta gactgtaaat gagttcttct taacttagct gtttccctac
1861 ttctgtgact tctgtgttag ccatcttatt tctttaaaat ctgagtcctg attggcttaa
1921 tgattttgca gcagacatgt ctccacatat tctcaaatgc tgtcatgcgg aaacgtatga
1981 aacagatgaa gaatgactga cccagatttt agatgtataa tgttgttaaa gtacatacta
2041 ctgtaaaaat atgggatgaa ttttatatat taagaaatgc caaaaacata gtttctgcac
2101 caagttaatt atccctgtcc tttcacattt atagggggaa aataaatact ttaatgttgt
2161 ttatagccta acagttattt gattttattc ttgcagaggg aatggaaagg aatggaaaga
2221 tttgttggcg taatttttga atatttgtta tgatcatatg aataagtaaa aaaattcatc
2281 ctgctgatgg cata
```

In some embodiments, the nucleic acid molecule of the present invention is included in a suitable vector. Typically, the vector is a viral vector, and more particularly an adeno-associated virus (AAV), a retrovirus, bovine papilloma virus, an adenovirus vector, a lentiviral vector, a vaccinia virus, a polyoma virus, or an infective virus. In some embodiments, the vector is an AAV vector. As used herein, the term “AAV vector” means a vector derived from an adeno-associated virus serotype, including without limitation, AAV1, AAV2, AAV3, AAV4, AAV5, AAV6, AAV7, AAV8, AAV9, and mutated forms thereof. AAV vectors can have one or more of the AAV wild-type genes deleted in whole or part, preferably the rep and/or cap genes, but retain functional flanking ITR sequences. Retroviruses may be chosen as gene delivery vectors due to their ability to integrate their genes into the host genome, transferring a large amount of foreign genetic material, infecting a broad spectrum of species and cell types and for being packaged in special cell- lines. In order to construct a retroviral vector, a nucleic acid encoding a gene of interest is inserted into the viral genome in the place of certain viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV 1, HIV 2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are known in the art, see, e.g.. U.S. Pat. Nos. 6,013,516 and 5,994,136, both of which are incorporated herein by reference. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest. Recombinant lentivirus capable of infecting a non-dividing cell wherein a suitable host cell is transfected with two or more vectors carrying the packaging functions, namely gag, pol and env, as well as rev and t at is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene into that packaging cell yields a producer cell which releases infectious viral particles carrying the foreign gene of interest. The env preferably is an amphotropic envelope protein which allows transduction of cells of human and other species. Typically, the nucleic acid molecule or the vector of the present invention include “control sequences′, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell. Another nucleic acid sequence, is a “promoter” sequence, which is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

By a “therapeutically effective amount” is meant a sufficient amount of the polypeptide or the nucleic acid molecule encoding thereof to prevent for use in a method for the treatment of the inflammatory disease at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

According to the invention, the polypeptide or the nucleic acid molecule of the present invention is administered to the subject in the form of a pharmaceutical composition. Typically, the polypeptide or the nucleic acid molecule (inserted or not into a vector) of the present invention may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, intradermal, transdermal, intrathecal and intranasal administration forms and rectal administration forms.

Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. Some variation in dosage will necessarily occur depending on the condition of the subject being treated. The person responsible for administration will, in any event, determine the appropriate dose for the individual subject.

In some embodiments, the TAFA4 polypeptide is administered in combination with classical treatment of inflammatory disease.

As used herein, the term “classical treatment” refers to any active agent used for the treatment of inflammatory disease.

In particular embodiment, the classical treatment of inflammatory disease refers to corticosteroids; aminosalicylates such as mesalamine, balsalazide and olsalazine; immunosuppressant drugs such as aziathioprine, mercaptopurine, cyclosporine and methotrexate; tumor necrosis factor (TNF)-alpha inhibitors such as infliximab, adalimumab and golimumab; aspirin, celecoxib; diclofenac; diflunisal; etodolac; ibuprofen; indomethacin; ketoprofen: ketorolac; nabumetone; naproxen; oxaprozin; piroxicam; salsalate; sulindac; tolmetin; antileukotrienes; antibiotic agents such as penicillin, quinoline, vancomycin, sulfonamides, ampicillin, ciprofloxacin, teicoplanin, telavancin, bleomycin, ramoplanin, decaplanin, chloramphenicol and sulfisoxazole.

As used herein, the terms “combined treatment”, “combined preparation”, “combined therapy” or “therapy combination” refer to a treatment that uses more than one medication. The combined therapy may be dual therapy or bi-therapy.

The medications used in the combined treatment according to the invention are administered to the subject simultaneously, separately or sequentially.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

The invention will be further illustrated by the following figures and examples. However, these examples and figures should not be interpreted in any way as limiting the scope of the present invention.

### FIGURES

FIG. 1: In vitro differentiation and polarization of human macrophages. A.

Experimental protocol used to generate polarized macrophage subsets from human purified monocytes. Monocytes were isolated from human blood and cultured with M-CSF (50 ng/ml) for six days to induce their differentiation in macrophages (My). The cells were then left untreated or treated for two days with specific factors to induce their polarization. Untreated cells remained MO My, IFN-γ (50 ng/ml)+LPS (10 ng/ml)-treated cells became M1 My, IL-4 (10 ng/ml)-treated cells became M2a My, and Dexamethasone (100 nM-1)-treated cells became M2c My. At the end of the culture, the different My types were harvested and used for analysis. B. Expression level (MFI) of different markers specific of My subsets: MO (CD14), M1 (CD80, CD86), M2a (CD86, CD206) and M2c (CD206, CD163). C. Polarized on unpolarized My subsets were cultured for one hour in presence of pHrodo zymosan bioparticles. Bioparticles phagocytosis (mean fluorescence intensity or 1VIFI) was assessed by flow cytometry. D. After the differentiation and polarization steps, My supernatants were harvested and the concentration of IL-6, IL-12p70, TNF-α and IL-10 was assessed by CBA in each individual subset. Data represent mean values ±S.E.M. of the data obtained for six donors. *P<0.05; **P<0.01; ***P<0.001; ****P<0.0001. One-way ANOVA with Tukey post-hoc test.

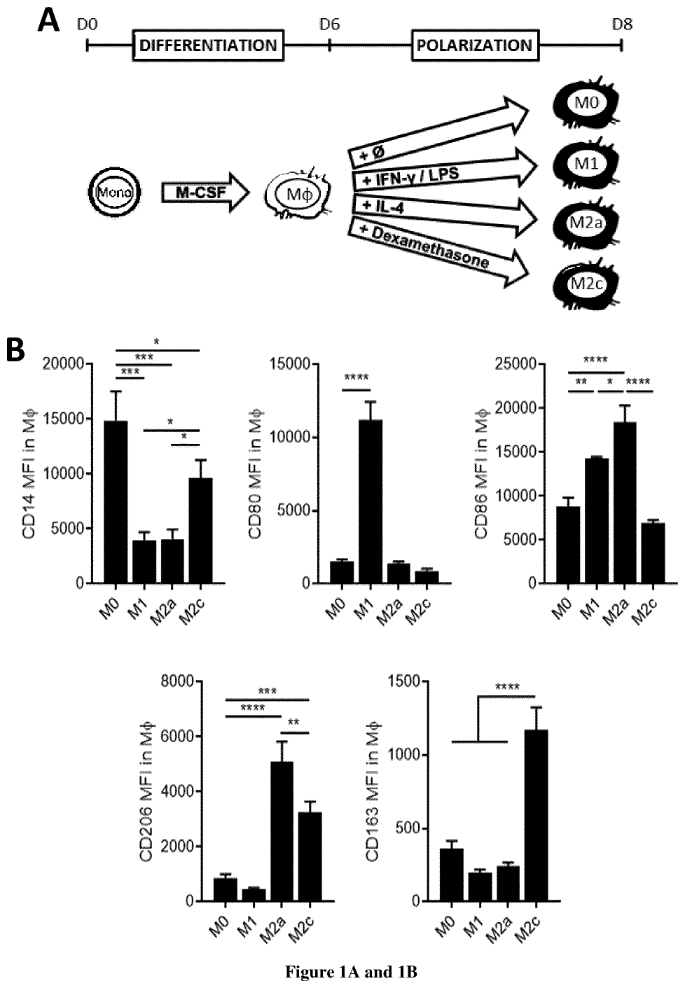

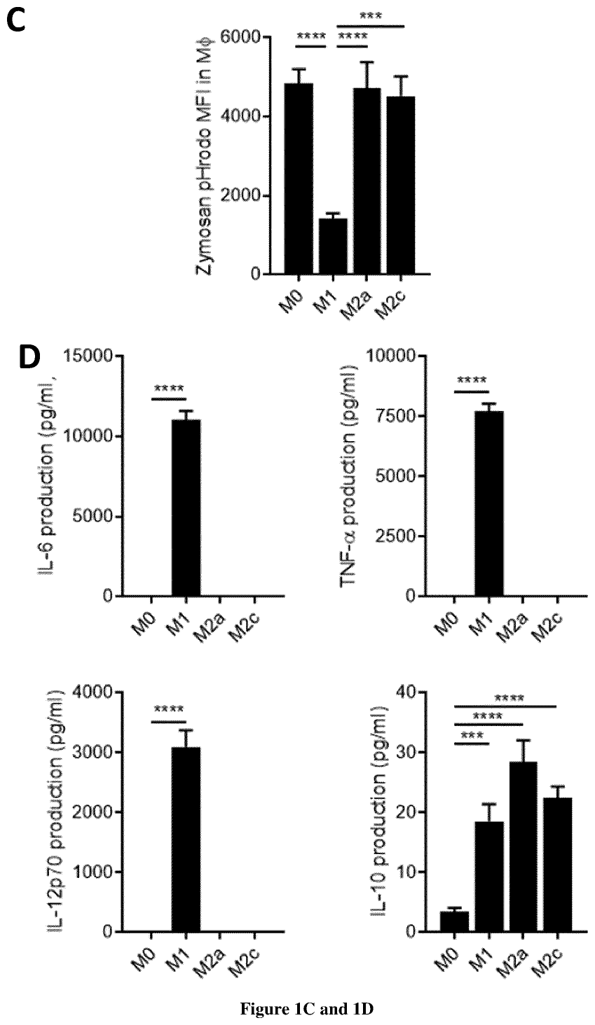

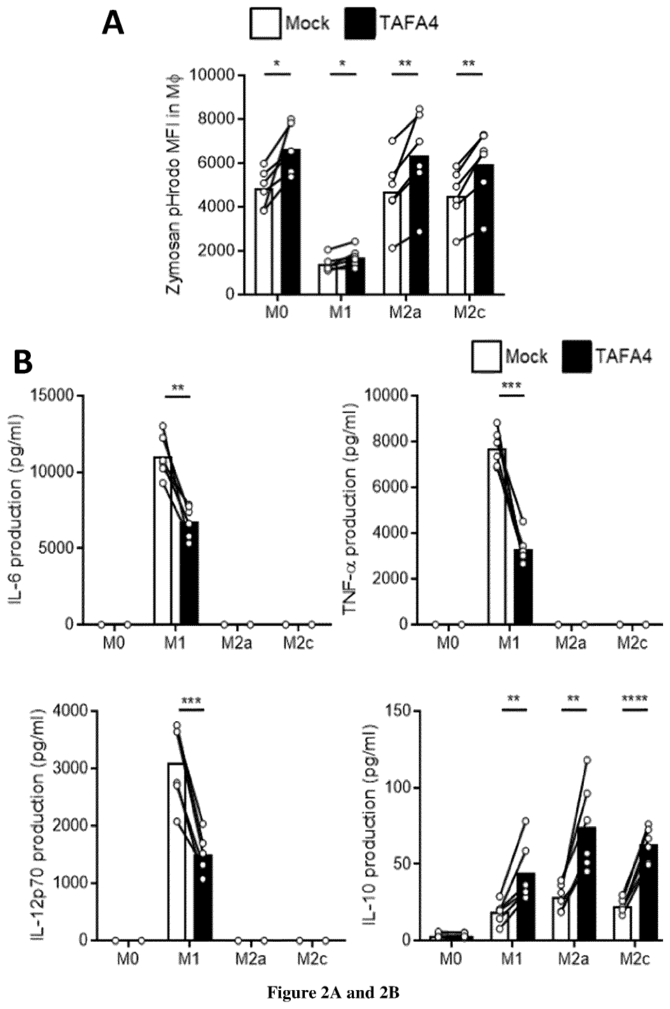

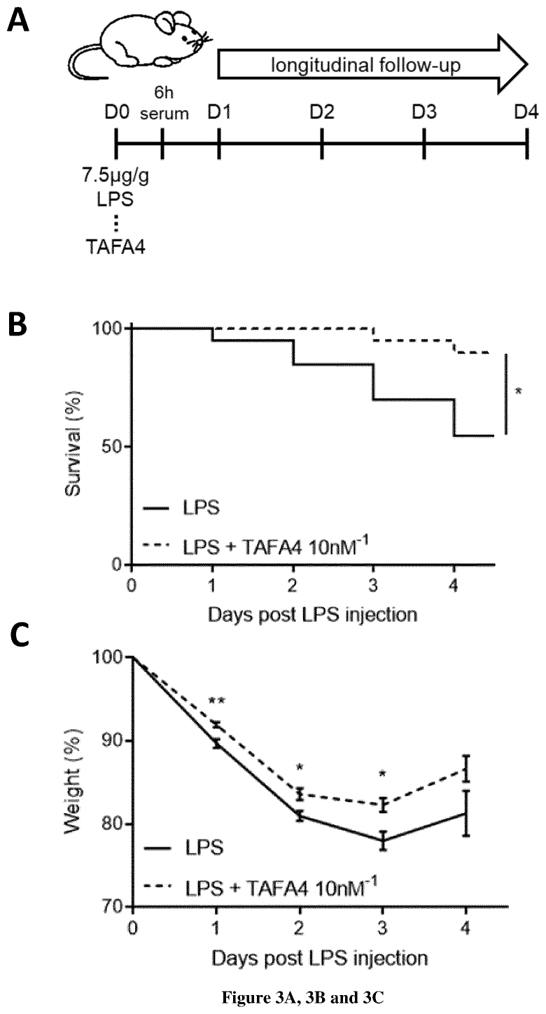

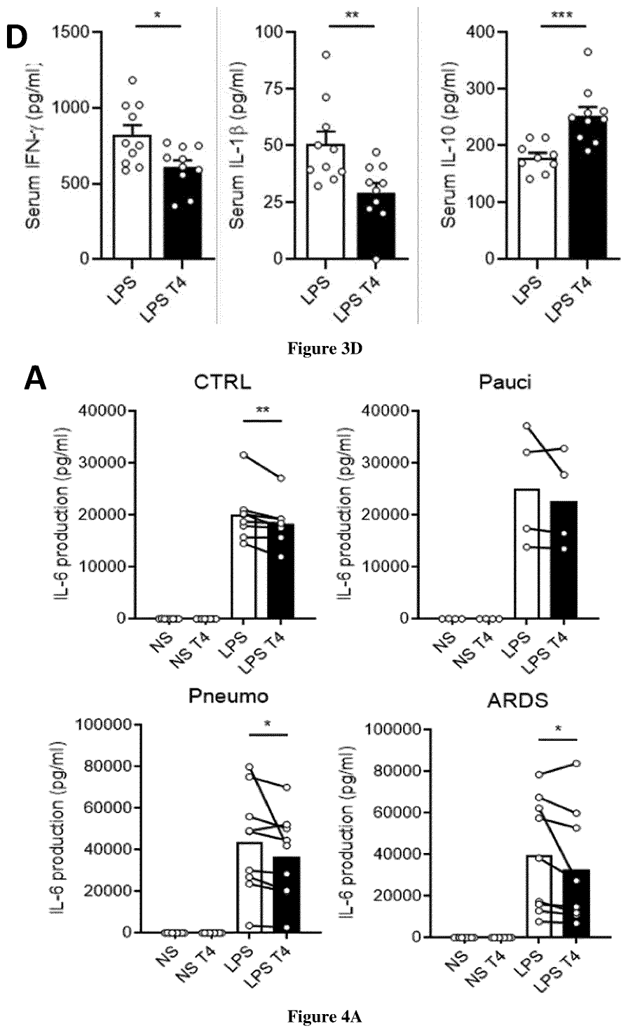

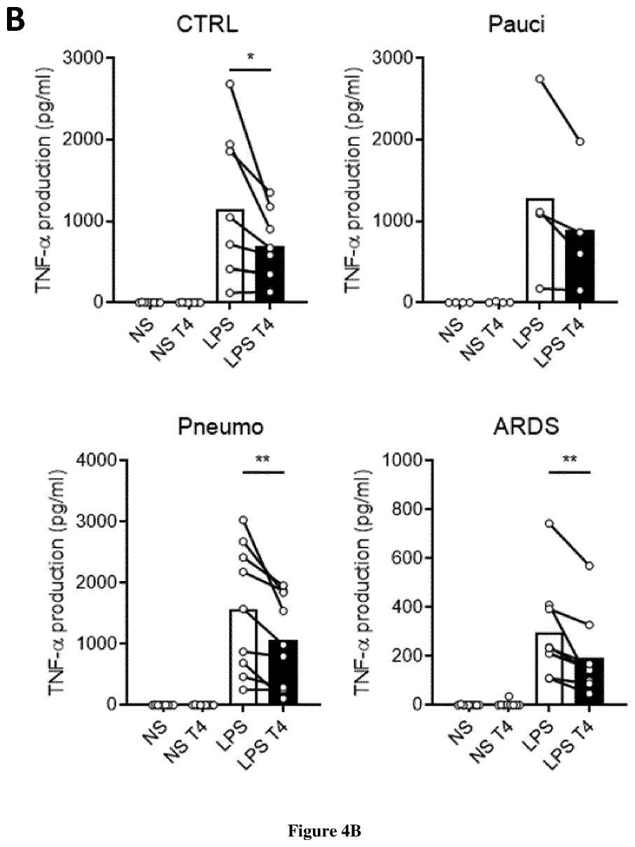

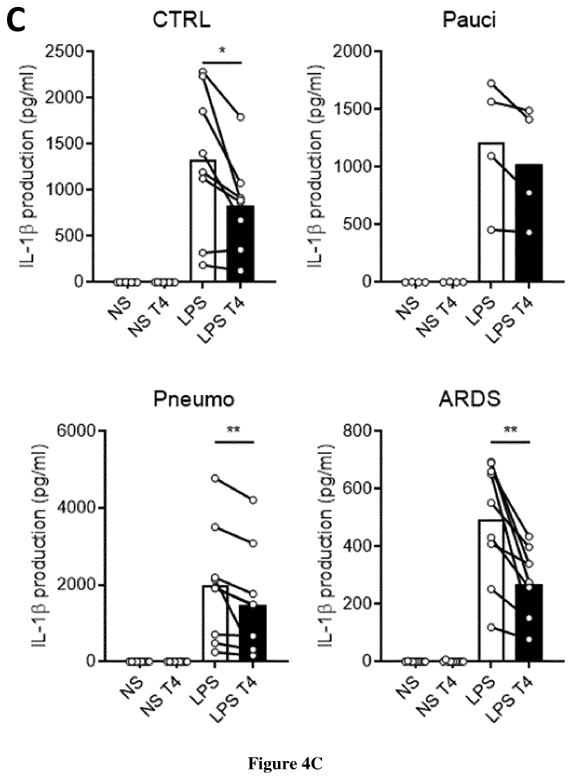

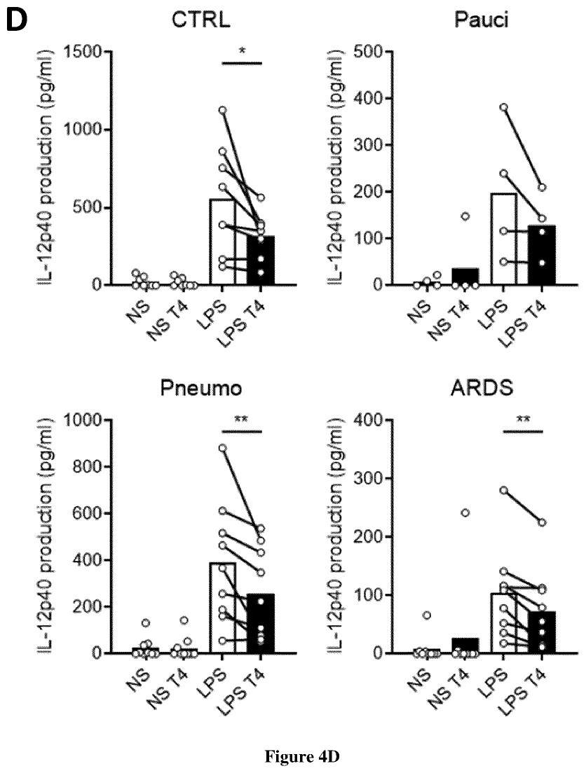

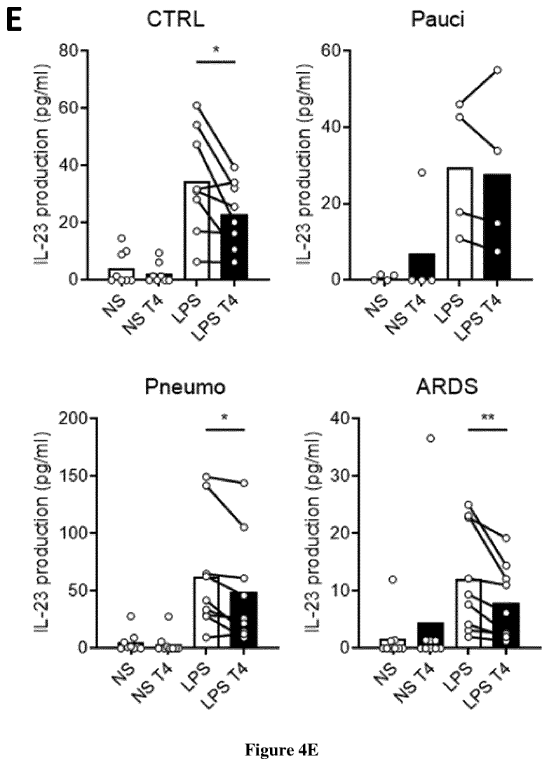

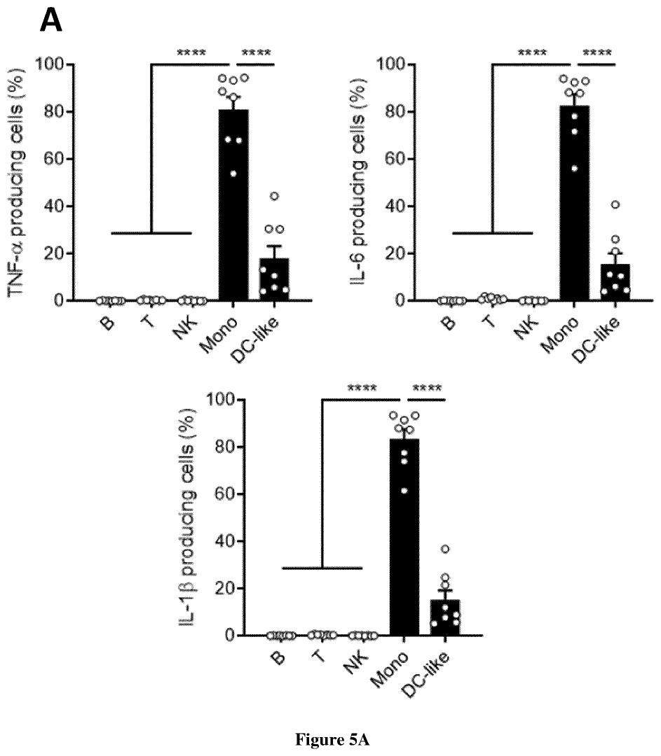

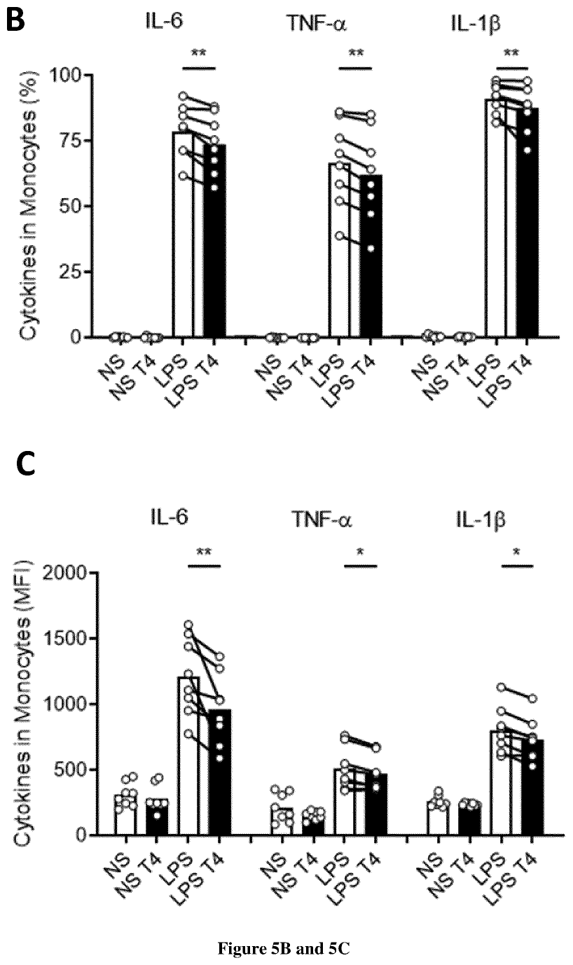

FIG. 2: TAFA4 promotes anti-inflammatory and pro-repair functions of human macrophages. Human macrophages were cultured in conditioning media inducing MO, Ml, M2a or M2c polarization as described in FIG. 1A. From day 6 to day 8, TAFA4 (500 ng/ml) or PBS (Mock) were added to the medium. A. On day 8, My subsets were incubated for one hour in presence of pHrodo zymosan bioparticles to assess their phagocytic functions by flow cytometry. Bioparticle phagocytosis (1W I) was analysed for untreated (in white) and TAFA4-treated (in black) My subsets. B. On day 8 of culture, the concentration of IL-6, IL-12p70, TNF-a, and IL-10 was assessed in the My culture supernatants by CBA. Data for untreated (in white) and TAFA4-treated (in black) conditions were then compared in each My subset. Data represent mean values ±S.E.M. of 6 independent donors. *P<0.05; **P<0.01; ***P<0.001; ****P<0.0001 (Paired t test).

FIG. 3: TAFA4 treatment protects mice from LPS-induced endotoxic shock. A.

Experimental protocol used for in vivo experiments. C57BL/6 male mice were i.p. injected with LPS (7.5 μg/g) at day 0 (DO), in the presence or absence of TAFA4 (10 nM⁻¹). Mouse sera were harvested six hours post-injection and mice were monitored for survival and weight changes during four days. B-C. Survival rate (%) (B) and weight evolution (% of initial weight) (C) of the mice injected with LPS, in absence (full line) or in presence (dotted line) of TAFA4 (10 nM⁻¹). n=20 mice per group from two independent experiments. D. Production of IFN-γ, IL-1(3 and IL-10 in the serum of mice injected with LPS, in the absence (in white) or in the presence of TAFA4 (in black). Data represent mean values±S.E.M. *P<0.05; **P<0.01; ***P<0.001; ****P<0.0001. Log-rank Mantel Cox test (B); multiple t test (C), Mann-Whitney test (D).

FIG. 4: TAFA4 inhibits inflammatory cytokine secretion and up-regulates IL-10 production in PBMC from COVID-19 patients. Blood samples were used to isolate peripheral blood mononuclear cells (PBMCs) from healthy donors (CTRL, n=8) and from SARS-CoV-2-infected patients with various forms of the disease: pauci: paucisymptomatic (n=4); Pneumo: mild pneumonia (n=9); and ARDS: severe pneumonia with Acute Respiratory Distress Syndrome (n=9). PBMCs were activated for 24 h with or without LPS (10Ong/ml, LPS or NS respectively), in the presence or absence of TAFA4 (500 ng/ml, T4 or 0 respectively). Concentrations of IL-6 (A), TNF-α (B), and IL-1(3 (C), IL-12p40 (D), and IL-23 (E) were measured in the supernatant of PBMCs. Data represent mean values±S.EM. *P<0.05; **P<0.01; ***P<0.001. Wilcoxon test.

FIG. 5: TAFA4 displays anti-inflammatory properties in monocytes of healthy donors. Intracellular staining and flow cytometry analysis of cytokine production in PBMC from healthy donors stimulated 6 hours with LPS (100 ng/ml). A. Relative frequency of immune cell subsets in IL-6-, TNF-α- or IL-IP-producing cells upon LPS stimulation. B. Percentages and C. mean fluorescence intensity (MFI) of IL-6, TNF-α and IL-1(3 expression in monocytes from PBMCs stimulated (LPS) or not (NS) and in the presence (T4) or not (0) of TAFA4 (500 ng/ml). Data represent mean values±S.E.M. *P<0.05; **P<0.01; ****P<0.0001. One-way ANOVA with Tukey's multiple comparison test (A); Wilcoxon test (B, C).

### Example

Material & Methods

Human blood samples

Over a period of one month (03-27-2020 to 04-24-2020), 22 subjects were recruited from three hospitals (Timone and North University Hospitals, and Laveran Military Hospital, Marseille). Nine of these patients were on mechanical ventilation for COVID-19-related-ARDS (P/F ratio <300) (ARDS group), nine patients required oxygen support at a rate of less than 5 1/min for COVID-19-related pneumonia (pneumonia group). Four patients had a paucisymptomatic form of COVID-19 compatible with outpatient care (paucisymptomatic group). COVID-19 was diagnosed on the basis of positive SARS-CoV-2 RT-PCR on nasopharyngeal samples and/or typical CT-scan findings. We also included eight healthy volunteers (control group), with no fever or symptoms in the days before sampling and negative for SARS-CoV-2 RT-PCR. Peripheral blood mononuclear cells (PBMC) were obtained from blood samples by density gradient centrifugation using Ficoll-Paque Plus (GE Healthcare). Freshly isolated PBMC were used for in vitro activations (FIGS. 4 and 5).

For the experiments on human macrophages (Mq) (FIGS. 1 and 2), blood buffy coat were obtained from the EFS (Etablissement Francais du Sang). PBMC were obtained from blood samples by density gradient centrifugation using Ficoll-Paque Plus (GE Healthcare) and used for macrophage differentiation.

Human Macrophage Differentiation and Polarization

Monocytes were isolated from PBMC using CD14⁺positive selection kit (Stemcell Technologies) according to the manufacturer protocol. Monocytes were then cultured in ImmunoCult-SF macrophage medium (Stemcell Technologies) with M-CSF (50 ng/ml, Stemcell Technologies) for six days to induce their differentiation in macrophages (My). Fresh medium has been added in the culture at day three or four. At day six, the cells were left untreated or treated for two days with specific factors to induce their polarization. Untreated cells remained MO My, IFN-γ (50 ng/ml, Stemcell Technologies)+LPS (10 ng/ml, from Escherichia coli strain 055:B5; Sigma Aldrich)-treated cells polarized into M1 My, IL-4 (long/ml, Stemcell Technologies)-treated cells polarized into M2a My, and Dexamethasone (100 nM⁻¹, Sigma Aldrich)-treated cells polarized into M2c My. At the end of the culture, macrophage supernatants were collected, centrifuged to remove cellular debris and frozen until quantification of the soluble factors. Then, the different My subsets were harvested using accutase (Stemcell Technologies) according to the manufacturer protocol and used for further experiments.

PBMC stimulation

Fresh PBMC (10⁶ cells) from control or SARS-CoV-2 infected patients were incubated in 24 well plates in 1 ml of complete medium composed of RPMI (Life Technologies) supplemented with 10% fetal calf serum (FCS, Life Technologies) and 1% antibiotics (penicillin/streptomycin, Life Technologies). Cells were then stimulated with 100 ng/ml lipopolysaccharide (LPS, from Escherichia coli strain 055:B5; Sigma-Aldrich) or mock-stimulated with PBS (Life Technologies) for 6 hours or 24 hours at 37° C. with 5% CO2. After 24h culture, the supernatant were collected, then centrifuged 5 minutes at 1500 rpm to remove floating cells and stored at −80° C. until quantification of the soluble factors. Alternatively, after six hours culture, cells were collected and used for intracellular staining.

TAFA4 Treatment of PBMC and Macrophages

Recombinant human TAFA4 (R&D Systems) was reconstituted according to manufacturer procedures and used on different cell types. For PBMC experiments, cells were treated either with TAFA4 (500 ng/ml) or PBS during the 24 hours of stimulation. For My experiments, cells were treated either with TAFA4 (500 ng/ml) or PBS during the two days of polarization. For the phenotypical and functional subsequent analyzes, TAFA4-treated cells were compared to PBS-treated cells from the same donor.

Macrophage Phenotypic Analysis

After differentiation and polarization, My were harvested, washed with PBS and stained with Live/Dead Fixable Blue Dead Cell Stain Kit (ThermoFisher Scientific) for 30 minutes at room temperature. Nonspecific binding was blocked using PBS supplemented with 10% human serum (Sigma Aldrich). Surface staining was then carried out in PBS supplemented with 1% human serum for 30 minutes at 4° C. The following antibodies were used: CD14-BUV737 (M5E2 clone), and CD8O-Pe-Cy7 (L307.4 clone; BD Technologies), HLA-DR-AF700 (L243 clone), CD86-PE (IT2.2 clone), CD206-Pe-Cy5 (15-2 clone) and CD163-BV510 (GHI/61 clone; BioLegend). Cell phenotype was characterized by multi-parameter flow cytometry analysis on a LSR-FORTESSA X20 cytometer (BD Bioscience). Data were further analyzed using FlowJo v10 software.

Phagocytosis Assay

The phagocytic activity of My in various polarizing conditions was measured as the cellular uptake of zymosan bioparticles. Cells were collected and incubated with 0.5 mg/ml pHrodo Zymosan Green BioParticles conjugate (Life Technologies) for 60 min at 37° C. Cells incubated with green pHrodo zymosan bioparticles at 4° C. were used as negative controls to confirm the specificity of the signal observed at 37° C. To stop the phagocytosis and remove the excess of bioparticles, cells were harvested and washed with cold PBS. Finally, the phagocytic activity was evaluated by flow cytometry using a LSR-FORTESSA X20 cytometer (BD Bioscience). Data were further analyzed using FlowJo v10 software.

Dosage of Human Cytokines and Chemokines

Cytokine production was assessed in culture supernatants through cytometric bead array (CBA, LEGENDplex, BioLegend), according to the manufacturer protocol. For PBMC stimulation experiments, the following soluble factors were tested: TNF-α, IL-1(3, IL-12p40, and IL-23. IL-6 production was assessed in the supernatants using CBA (BD Technologies) after a hundred-fold dilution. For My experiments, the following soluble factors were tested: TNF-α, IL-6, IL-12p70, and IL-10. Data were then acquired using a FACSCanto II cytometer (BD Bioscience, for PBMC experiments) or a LSR-FORTESSA X20 cytometer (BD Bioscience, for My experiments). Results were then analyzed using LEGENDplex v8 software (BioLegend) or FCAP array v3 software (BD Technologies).

Intracellular Staining in PBMC

PBMC were stimulated for 6 hours in the presence or not of LPS (100 ng/ml, from Escherichia coli strain 055:B5; Sigma-Aldrich) and TAFA4 (500 ng/ml; R&D Systems). GolgiPlug (BD Technologies) was added in each well according to the manufacturer procedures. Cells were then collected and stained with Live/Dead Fixable Near-IR Dead Cell Stain Kit (ThermoFisher Scientific) for 30 minutes at room temperature. Surface staining was then carried out in PBS supplemented with mouse serum for 30 minutes at 4° C. The following antibodies were used: CD45-V500 (HI30 clone), CD16-BUV496 (3G8 clone), CD56-BUV395 (B159 clone) and CD3-BUV737 (UCHT1 clone; BD Technologies), HLA-DR-AF700 (L243 clone), CD19-BV711 (HIB19 clone), and CD11b-BV785 (ICRF44 clone; BioLegend). Intracellular staining was then performed using Cytofix/Cytoperm kit (BD Technologies) according to the manufacturer protocol. The following antibodies were used: TNF-APC (MAb 11 clone), IL-10-FITC (AS10 clone) and IL-6-PE (AS12 clone; BD Technologies). Cell phenotype was characterized by multi-parameter flow cytometry analysis on a LSR-FORTESSA X20 cytometer (BD Bioscience). Data were further analyzed using FlowJo v10 software.

Mouse Experiments

C57BL/6J mice were purchased from Janvier Lab. All mice were maintained under specific-pathogen—free conditions in the Centre d′Immunologie de Marseille Luminy. Mice were housed under a standard 12 h/12 h light—dark cycle with food and water ad libitum. Nine-week-old male mice were used for all experiments. LPS (from Escherichia coli strain 055:B5; Sigma Aldrich) diluted in PBS was injected intraperitoneally (7.5 μg per gram mouse body weight). Mice were injected either with LPS alone or with LPS+TAFA4 (10 nM⁻¹, R&D Systems). All LPS injections were performed between 9 a.m. and 10 a.m., and mice were checked every 24 hours for body weight, survival, and signs of distress until day 4. For IL-10 blockade experiments, mice were intraperitoneally injected with 500 μg anti—IL-10 (JES5-2A5), or rat IgG1 (HRPN; all from BioXCell) twenty-four hours before LPS injection.

Blood samples were obtained using retro-orbital blood collection at six hours post LPS-treatment, and processed to eliminate red blood cells. The same mice were used for blood collection and longitudinal follow-up. Serum samples were then stored at −80° C. until quantification of soluble mediators. The concentrations of various soluble factors was assessed by CBA according to the manufacturer's protocol (BD Biosciences). All samples were diluted by half before quantification.

Statistical Analyses

Statistical analyses were performed using Graphpad Prism v8 software. Data represent mean values ±standard error of mean (S.E.M.) and the number of independent donors/mice/experiments is indicated in each figure legend. Paired data were compared with two-tailed paired t tests if the values followed a Gaussian distribution or Wilcoxon test otherwise. Unpaired data were compared with two-tailed Student's t test if the values followed a Gaussian distribution or Mann—Whitney test otherwise. For multiple comparisons, we used one-way Kruskal-Wallis with Dunn's multiple comparison test, analysis of variance (ANOVA) with Tukey post-hoc test or multiple t tests. Differences in survival were evaluated with the Mantel—Cox test. The statistical test used for each panel is stated in figure legend. Data were considered statistically significant if the P value obtained was lower than 0.05. *P<0.05; **P<0.01; ***P<0.001; ****P<0.0001.

Results

TAFA4 promotes human macrophage anti-inflammatory and pro-repair functions.

We analysed the role of TAFA4 on human macrophages phenotype and functions. We isolated peripheral blood mononuclear cells (PBMCs) from healthy donors and sorted CD14⁺monocytes. These monocytes were in vitro differentiated into macrophages (Mq) in the presence of M-CSF during 6 days. Different conditioning media were then used during two additional days to polarize these macrophages in different subtypes as described in FIG. 1A. For each donor, we generated unpolarized My (MO), pro-inflammatory My (M1), pro-repair My (M2a) and regulatory My (M2c). To validate this protocol of macrophage differentiation, we analyzed the phenotype of the different subsets by flow cytometry. We analyzed the expression level of CD14, CD80, CD86, CD206 and CD163 to establish the phenotypic signature of each My subset (FIG. 1B). As expected, compared to the polarized My subsets, MO My expressed high levels of CD14 but low levels of the other markers. M1 My expressed high levels of CD80 and CD86 but low level of CD14. M2a My expressed high levels of CD206 and CD86 but low CD14 levels. Finally, M2c My expressed high levels of CD206 and CD163 with intermediate CD14 levels.

We then used this in vitro model to assess the effect of TAFA4 on My phagocytic and cytokine production functions. After 6 days of differentiation in M-CSF, My were cultured in conditioning media to induce MO, Ml, M2a or M2c polarization in the presence or in absence (Mock) of TAFA4 (500 ng/ml). We first assessed the phagocytic capacities of the macrophages subtypes by using pHrodo zymosan bioparticles that become fluorescent after internalization and processing in acid compartments (phagosomes). After one hour of culture with the bioparticles, My were harvested and their fluorescence, reflective of their phagocytic ability, was analyzed by flow cytometry. We observed that M1 inflammatory My display reduced phagocytic functions compared to MO, M2a or M2c cells (FIG. 1C). Moreover, TAFA4 induced a significant increase of the phagocytic function in all the macrophage subsets (FIG. 2A). We then analyzed the effect of TAFA4 on the cytokines produced by the My subtypes. We analyzed the production of IL-6, TNF-α, IL-12p70, and IL-10 in the culture supernatants (on day 8) by Cytometric Bead Array (CBA). As expected, we observed that M1 macrophages produced high levels of pro-inflammatory cytokines IL-6, TNF-α, and IL-12p70 (FIG. 1D) whereas M2a and M2c Mq only produced IL-10. TAFA4 modified the profile of cytokine production by macrophages towards a more anti-inflammatory profile. In the supernatant of M1 My, we observed a drastic decrease in the production of pro-inflammatory cytokines, associated with an increased production of the anti-inflammatory cytokine IL-10 (FIG. 2B). Moreover, TAFA4 also up-regulated IL-10 production by M2a and M2c

In conclusion, these data demonstrate that the neuropeptide TAFA4 promotes the anti-inflammatory and pro-repair functions of human macrophages. TAFA4 can act on all the subtypes of macrophages by increasing their production of IL-10 and reinforcing their phagocytic capacities. Moreover, TAFA4 limits the pro-inflammatory cytokine production of M1 macrophages.

TAFA4 Treatment Protects Mice from LPS-Induced Endotoxic Shock.

The human data described above suggest a potential beneficial role of TAFA4 in inflammatory diseases. To test the anti-inflammatory role of TAFA4 in vivo, we used an animal model of systemic inflammation in which a cytokine storm jeopardizing host survival is induced. We injected LPS into mice to induce an endotoxic shock (FIG. 3A). In this model, classically used to mimic sepsis, TAFA4 treatment increased mouse survival (FIG. 3B). Moreover, mice treated with TAFA4 had a reduced weight loss (FIG. 3C). This higher survival rate and lower disease severity were associated lower serum levels of the inflammatory cytokines IFN-γ and IL-1(3 (FIG. 3D). By contrast, IL-10 levels were higher than those in untreated mice (FIG. 3D). Finally, we showed that in this model the main effect of TAFA4 on mouse resistance to endotoxic shock is mediated through its ability to induce IL-10. Indeed, the survival of mice treated with anti-IL-10 neutralizing antibodies, was no longer improved by TAFA4 treatment. However, even in the absence of IL-10, TAFA4 continued to have an effect on reducing serum levels of other inflammatory cytokines/chemokines such as TNF-α, IL-12, CCL3, CCL-4 and CXCL9 (data not shown), showing that TAFA4 can reduce inflammation by controlling multiple pathways.

These results provide a proof of concept of the potent anti-inflammatory properties of TAFA4 in vivo and demonstrate that TAFA4 treatment can protect mice from an inflammatory disease.

TAFA4 Acts on Human Monocytes and Reduces Inflammatory Cytokine Production by PBMCs of SARS-CoV-2 Infected Patients.

SARS-CoV-2 infection can lead to severe forms of COVID-19 characterized by a cytokine storm and the invasion of the lungs by overactivated myeloid cells. In some patients, this disease can lead to severe tissue damage resulting in lung fibrosis and death. In this context and based on our previous results, we hypothesized that treatment with the neuropeptide TAFA4 might be beneficial to treat this disease and other inflammatory pathologies. We therefore studied the anti-inflammatory role of TAFA4 in a cohort of 22 patients infected with SARS-CoV-2. Peripheral blood mononuclear cells (PBMCs) from COVID-19 patients with various forms of the disease (paucisymptomatic, mild pneumonia, severe pneumonia with acute respiratory distress syndrome: ARDS) were activated for 24 hours with LPS in the presence or absence of TAFA4. We showed that TAFA4 reduced the production of the inflammatory cytokines IL-6, TNF-α, IL-1(3, IL-12p40 and IL-23 by PBMC from healthy donors and COVID-19 patients, including patients with ARDS (FIG. 4A-D)

We also performed intracellular staining and multi-parametric flow cytometry analyses to identify the cellular source of IL-6, TNF-α, and IL-1(3 in the PBMC. We then monitored cytokine expression in B cells (CD19⁺), T cells (CD3⁺), NK cells (CD56⁺CD16^{+/-}CD3⁻), monocytes (CD11b⁺HLA-DR⁺) and DC-like cells (CD1 lb⁻I-ILADR⁺). We found that the cytokine-producing cells were mainly monocytes (85%), and DC-like cells to a lower extend (15%) (FIG. 5A). On the contrary, B, T, and NK cells did not produced cytokines after LPS stimulation. Interestingly, we observed that upon TAFA4 treatment, LPS-activated monocytes displayed a significant reduction in their cytokine production both in term frequency (% of producing cells) and quantity (MFI), compared to untreated cells (FIG. 5B-C). These results are consistent with the endogenous anti-inflammatory and pro-repair role of TAFA4 described in our previous study (Hoeffel et al. in revision).

We then focused our studies on M2c macrophages, which are differentiated in vitro in the presence of glucocorticoids and represent a type of anti-inflammatory cells that promotes tissue repair and could be beneficial in inflammatory diseases. Such M2c-like macrophages, expressing the marker CD163, were already observed in the lung of COVID-19 patients (data from the Vivier's lab: Carvelli et al. In press, Nature). We performed RNA-seq analysis of M2c macrophages differentiated in the presence or absence of TAFA4, and showed that TAFA4 reduces the expression of genes involved in the inflammatory response such as type I and II interferons, and also in the migration of leukocytes to the inflammatory site (data not shown). On the contrary, TAFA4 increases the production of genes involved in the maintenance of homeostasis, and corticosteroid response. The interest of these data is reinforced by results from the RECOVERY trial showing that treatment with corticosteroids improves the clinical course of COVID-19 patients and reduced deaths by one-third in most severe patients (https://www.ox.ac.uk/news/2020-06-16-low-cost-dexamethasone-reduces-death-one-third-hospitalised-pati ents- severe).

Collectively, these results demonstrate that the neuropeptide TAFA4 can reprogram human monocytes and macrophages toward an anti-inflammatory phenotype. TAFA4 can act on PBMC from COVID-19 patients at any stage of the disease. TAFA4 can also protect from the deleterious effects of over-inflammation in vivo in a mouse preclinical model of acute inflammatory disease. These data strongly support the use of TAFA4 as a therapeutic agent in inflammatory diseases, including sepsis or COVID-19, to reduce the activation and tissue recruitment of immune cells.

### REFERENCES

Throughout this application, various references describe the state of the art to which this invention pertains. The disclosures of these references are hereby incorporated by reference into the present disclosure.
1. D.M. Mosser, J.P. Edwards, Exploring the full spectrum of macrophage activation, Nat. Rev. Immunol. 8 (2008) 958-969.
2. Y.R Na, S. Je, S.H Seok, Metabolic features of macrophages in inflammatory diseases and cancer. Cancer Letters 413 (2018) 46-58
3. I.B. McInnes, G. Schett, The pathogenesis of rheumatoid arthritis, N. Engl. J. Med. 365 (2011) 2205-2219.
4. K.J. Moore, F.J. Sheedy, E.A. Fisher, Macrophages in atherosclerosis: a dynamic balance, Nat. Rev. Immunol. 13 (2013) 709-721.
5. Tom Tang, Y. et al. TAFA: a novel secreted family with conserved cysteine residues and restricted expression in the brain. Genomics 83, 727-734, doi:10.1016/j.ygeno.2003.10.006 (2004).
6. Delfini, M. C. et al. TAFA4, a chemokine-like protein, modulates injury-induced mechanical and chemical pain hypersensitivity in mice. Cell Rep 5, 378-388, doi:10.1016/j.celrep.2013.09.013 (2013).
7. Wang, W. et al. FAM19A4 is a novel cytokine ligand of formyl peptide receptor 1 (FPR1) and is able to promote the migration and phagocytosis of macrophages. Cellular & molecular immunology 12, 615-624, doi:10.1038/cmi.2014.61 (2015).
8. Chan, J.F.; Yuan, S.; Kok, K.H.; To, K.K.; Chu, H.; Yang, J.; Xing, F.; Liu, J.; Yip, C.C.; Poon, R. W., et al. A familial cluster of pneumonia associated with the 2019 novel coronavirus indicating person-to-person transmission: a study of a family cluster. Lancet 2020,
9. Huang, C.; Wang, Y.; Li, X.; Ren, L.; Zhao, J.; Hu, Y.; Zhang, L.; Fan, G.; Xu, J.; Gu, X., et al. Clinical features of patients infected with 2019 novel coronavirus in Wuhan, China. Lancet 2020, 395, 497-506
10. Lu, R.; Zhao, X.; Li, J.; Niu, P.; Yang, B.; Wu, H.; Wang, W.; Song, H.; Huang, B.; Zhu, N., et al. Genomic characterisation and epidemiology of 2019 novel coronavirus: implications for virus origins and receptor binding. Lancet 2020, 395, 565-574.
11. Cao X. COVID-19: immunopathology and its implications for therapy. Nature Reviews. 2020; doi:10.1038/s41577-020-0308-3

1. A method of treating an inflammatory disease in a subject in need thereof comprising administering to the subject a therapeutically effective amount of a TAFA4 polypeptide or a nucleic acid molecule encoding thereof.

2. The method of claim 1 wherein the inflammatory disease is selected from the group consisting of allergy, asthma, preperfusion injury, transplant rejection, sepsis, septic shock, arthritis, rheumatoid arthritis, acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), x-linked hyper IgM syndrome, sclerosis, systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis, inflammatory bowel disease (IBD), Crohn's disease, colitis, ulcerative colitis, colitis ulcerosa, microscopic colitis, collagenous colitis, colitis polyposa, necrotizing enterocolitis, transmural colitis, autoimmune inflammatory bowel disease, pyoderma gangrenosum, erythema nodosum, primary sclerosing cholangitis, episcleritis, respiratory distress syndrome, adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis, Rasmussen's encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN), idiopathic membranous GN or idiopathic membranous nephropathy, membrano- or membranous proliferative GN (A/PGN), rapidly progressive GN, allergic conditions, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis, lymphomatoid granulomatosis, Wegener's granulomatosis, agranulocytosis, vasculitides, including vasculitis, large vessel vasculitis, polymyalgia rheumatica, giant cell (Takayasu's) arteritis, medium vessel vasculitis, Kawasaki's disease, polyarteritis nodosa, microscopic polyarteritis, CNS vasculitis, necrotizing, cutaneous, hypersensitivity vasculitis, systemic necrotizing vasculitis, and ANCA-associated vasculitis, such as Churg-Strauss vasculitis or syndrome (CSS), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigus, optionally pemphigus vulgaris, pemphigus foliaceus, pemphigus mucus-membrane pemphigoid, pemphigus erythematosus, autoimmune polyendocrinopathies, Reiter's disease or syndrome, immune complex nephritis, antibody-mediated nephritis, neuromyelitis optica, polyneuropathies, chronic neuropathy, IgM polyneuropathies, IgM-mediated neuropathy, thrombocytopenia, thrombotic thrombocytopenic purpura (TTP), idiopathic thrombocytopenic purpura (ITP), autoimmune orchitis and oophoritis, primary hypothyroidism, hypoparathyroidism, autoimmune thyroiditis, Hashimoto's disease, chronic thyroiditis (Hashimoto's thyroiditis); subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis, allergic encephalomyelitis, experimental allergic encephalomyelitis (EAE), myasthenia gravis, thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AGED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis, optionally benign monoclonal gammopathy or monoclonal gammopathy of undetermined significance, MGUS, peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia greata, CREST syndrome (calcinosis, Raynaud's phenomenon, esophageal dysmotility, sclerodactyl), and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillo sis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis (e.g. chronic pancreatitis), polyradiculitis acuta, pyoderma gangrenosum, Quervain's thyreoiditis, acquired splenic atrophy, infertility due to antispermatozoan antobodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, nonalcoholic fatty liver disease and endometriosis.

3. The method of claim 1 wherein the inflammatory disease is sepsis.

4. The method of claim 1, wherein the inflammatory disease is a viral infection which caused lung inflammation.

5. The method of claim 4, wherein the viral infection is severe acute respiratory syndrome.

6. The method of claim 4, wherein the viral infection is Covid-19.

7. The method of claim 1 wherein the TAFA4 polypeptide comprises a sequence having at least 90% sequence identity to the sequence as set forth in SEQ ID NO: 1.

8. The method of claim 1 wherein the TAFA4 polypeptide is fused to an immunoglobulin constant domain to constitute an immunoadhesin.

9. The method of claim 1 wherein the nucleic acid molecule is included in a suitable vector, such as viral vector (e.g. AAV).

10. The method of claim 1 wherein he TAFA4 polypeptide or the nucleic acid molecule encoding thereof the TAFA4 polypeptide is administered with another active agent used for the treatment of inflammatory disease.
